(19) United States
(12) Patent Application Publication
(10) Pub. No.: US 2025/0222256 A1
(43) Pub. Date: Jul. 10, 2025

(54) Tissue Inflammation and Electrical Stimulation

(71) Applicant: Neurostim Technologies LLC, Wellesley, MA (US)

(72) Inventors: Constance CARDASIS, Putnam, CT (US); Ann G. YEE, Sharon, MA (US); William C. ALTMANN, Austin, TX (US); Hoo-min D. TOONG, Cambridge, MA (US)

(21) Appl. No.: 19/013,307

(22) Filed: Jan. 8, 2025

Related U.S. Application Data
(60) Provisional application No. 63/618,444, filed on Jan. 8, 2024.

(51) Int. Cl. A61N 1/36; A61N 1/04

(52) U.S. Cl. CPC A61N 1/36021; A61N 1/0456; A61N 1/0492; A61N 1/36031

(57) ABSTRACT

Embodiments treat inflammation of a user by applying a first patch on a dermis of the user adjacent to an afferent nerve, the first patch comprising first electrodes, and applying a second patch on the dermis of the user adjacent to a vagus nerve or a vagus nerve connecting branch, the second patch comprising second electrodes. Embodiments generate a treatment protocol during a time period comprising simultaneously applying a first electrical stimuli to the afferent nerve via the first electrodes of the first patch and applying a second electrical stimuli to the vagus nerve or the vagus nerve connecting branch via the second electrodes of the second patch.

### CROSS REFERENCE TO RELATED APPLICATIONS

This application claims priority to U.S. Provisional Patent Application Ser. No. 63/618,444, filed on Jan. 8, 2024, the disclosure of which is hereby incorporated by reference.

### FIELD

Embodiments are directed generally to systems and methods for stimulating tissues in humans.

### BACKGROUND INFORMATION

Chronic inflammation underlies damage to all internal body systems and affects the quality of life. Medications, such as non-steroidal anti-inflammatory drugs (NSAIDs), and steroid drugs, have side effects.

Chronic inflammation has been shown to underlie many chronic diseases, including, arthritis, diabetes, obesity and chronic metabolic syndrome, cirrhosis, inflammatory bowel disease, auto-immune diseases, asthma, Alzheimer's and others.

### BRIEF DESCRIPTION OF THE DRAWINGS

FIG. 1 shows a TNA device/patch applied to the tibial nerve at the ankle in accordance to embodiments.

FIG. 2 shows several mechanisms by which the nervous and immune system interact.

FIG. 3 shows the linkage of incoming information from both somatic and autonomic pathways to higher brain centers.

FIG. 4 shows the noradrenergic reflex system inhibiting inflammation.

FIG. 5A shows the site of electrical stimulation of the common peroneal nerve in the lower leg, to inhibit inflammation in accordance to embodiments.

FIG. 5B shows the site of electrical stimulation of the auricular branch of the vagus nerve to inhibit inflammation in accordance to embodiments.

FIG. 6A shows a TNA Device System applied to the skin, with one Paired TNA Device, and two Paired Sensor Devices, each applied on top of the Inflammation Site, in accordance to embodiments.

FIG. 6B shows a Smart TNA Device, with a securing mechanism, and one or more electrode pairs with each pair having a positive electrode and a negative electrode, and a power source, and a processor, and a Sensor, in accordance to embodiments.

FIG. 6C shows a Paired TNA Device, with a securing mechanism, and one or more electrode pairs with each pair having a positive electrode and a negative electrode, and a power source, and a processor, in accordance to embodiments.

FIG. 7 shows a User with a Paired TNA Device, and a Smart TNA Device in accordance to embodiments.

FIG. 8 shows the human ear in detail, with the Non-Auricular Vagus Nerve Stimulation Sites (taVNS) and the Auricular Vagus Nerve Stimulation Sites (staVNS).

### DETAILED DESCRIPTION

Embodiments are directed to a system and method for treating inflammation using electrical stimulation. Inflammation may be categorized as systemic, affecting wide areas of the body, and local, affecting a small area of tissue. Different physiological mechanisms are at work modulating the inflammatory process. The neuroimmune system involves a complex series of interactions between autonomic and somatic neural systems, hormonal systems, and the immune system. Embodiments use these pathways and external electrical stimulation to limit the effects of inflammation. Embodiments use two or more external devices simultaneously (on an afferent nerve local to the inflammation and on a vagus nerve or one or more vagus nerve connecting branches) in order to provide an enhanced anti-inflammatory response.

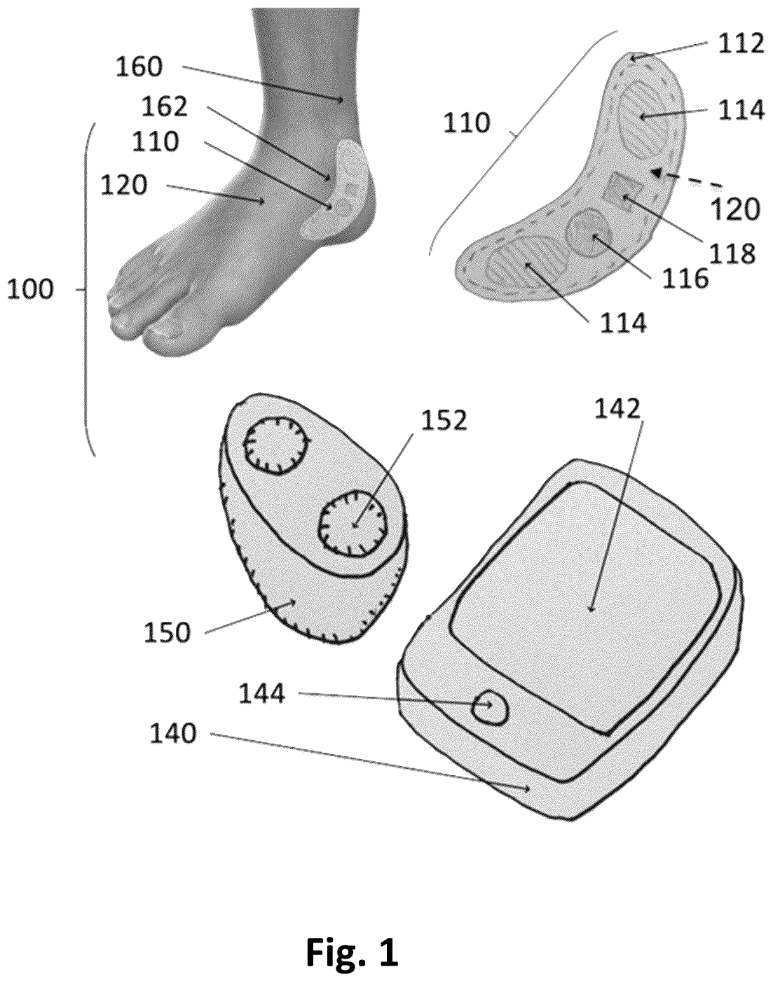

FIG. 1 shows a User 120 with a TNA System 100, including a Topical Nerve Activator (TNA) Device (or “Patch”) 110, with a securing mechanism 112, and one or more electrode pairs 114 with each pair having a positive electrode and a negative electrode, and a power source 116, and a processor 118, and a Sensor 120; and an optional Smart Controller 140 (e.g., a fob or smartphone), with a display 142, and an acknowledgment button 144; and an optional Fob 150 with one or more buttons 152 in accordance to embodiments. FIG. 1 shows the TNA Device 110 affixed to the skin at the Tibial Nerve 160 on the right or left Ankle 162.

Patch 110 is used to stimulate nerves and is convenient, unobtrusive, self-powered, and may be controlled from a smartphone or other control device. This has the advantage of being non-invasive, controlled by users themselves, and potentially distributed over the counter without a prescription. Patch 110 provides a means of stimulating nerves without penetrating the dermis, and can be applied to the surface of the dermis at a location appropriate for the nerves of interest. In examples, patch 110 is applied by the user and is disposable.

Patch 110 in examples can be any type of device that can be fixedly attached to a user, using adhesive in some examples, and includes a processor/controller and instructions that are executed by the processor, or a hardware implementation without software instructions, as well as electrodes that apply an electrical stimulation to the surface of the user's skin, and associated electrical circuitry. Patch 110 in one example provides topical nerve activation/stimulation on the user to provide benefits to the user, including treatment for inflammation.

Patch 110 in one example can include a flexible substrate, a malleable dermis conforming bottom surface of the substrate including adhesive and adapted to contact the dermis, a flexible top outer surface of the substrate approximately parallel to the bottom surface, one or more electrodes positioned on the patch proximal to the bottom surface and located beneath the top outer surface and directly contacting the flexible substrate, electronic circuitry (as disclosed herein) embedded in the patch and located beneath the top outer surface and integrated as a system on a chip that is directly contacting the flexible substrate, the electronic circuitry integrated as a system on a chip and including an electrical signal generator integral to the malleable dermis conforming bottom surface configured to electrically activate the one or more electrodes, a signal activator coupled to the electrical signal generator, a nerve stimulation sensor that provides feedback in response to a stimulation of one or more nerves, an antenna configured to communicate with a remote activation device, a power source in electrical communication with the electrical signal generator, and the signal activator, where the signal activator is configured to activate in response to receipt of a communication with the activation device by the antenna and the electrical signal generator configured to generate one or more electrical stimuli in response to activation by the signal activator, and the electrical stimuli configured to stimulate one or more nerves of a user wearing patch 110 at least at one location proximate to patch 110. Additional details of examples of patch 100 beyond the novel details disclosed herein are disclosed in U.S. Pat. No. 10,016,600, entitled “Topical Neurological Stimulation”, the disclosure of which is hereby incorporated by reference.

### Inflammation Mechanisms

Several of the interactions between the nervous system and the immune system are shown in inflammatory cytokines, pattern recognition products and other inflammatory molecules activate somatic and autonomic fibers. These initiate several pathways, including: 1) a neurohormonal pathway; 2) a cholinergic pathway; 3) a noradrenergic pathway; and 4) an axonal response involving no synapses.

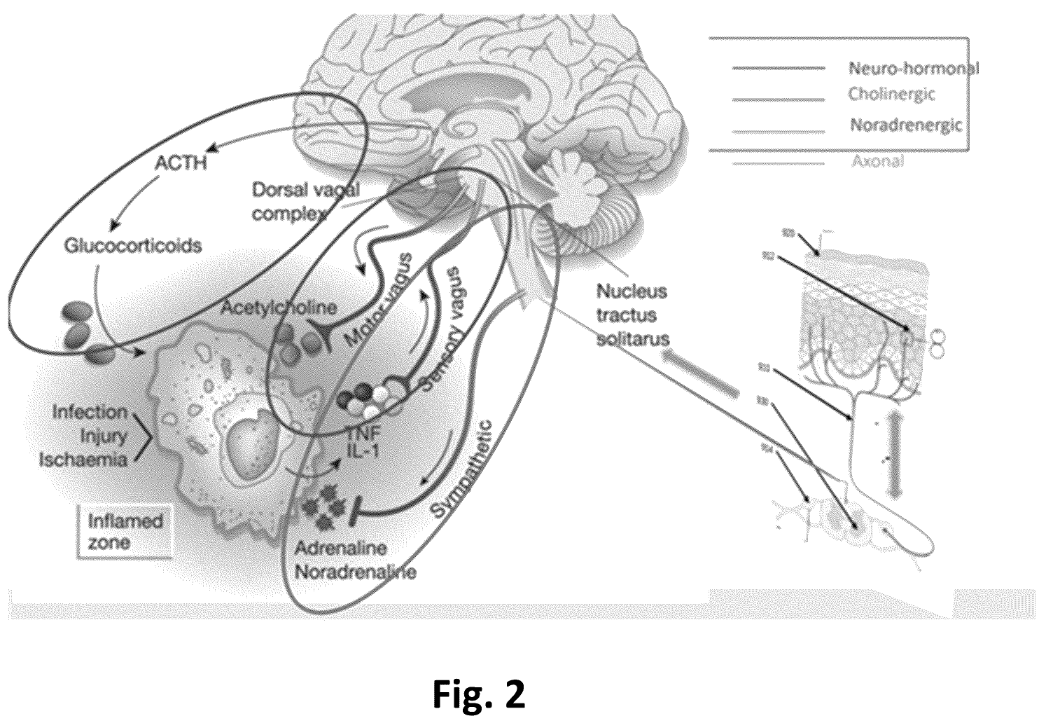

FIG. 2 shows several mechanisms by which the nervous and immune system interact.

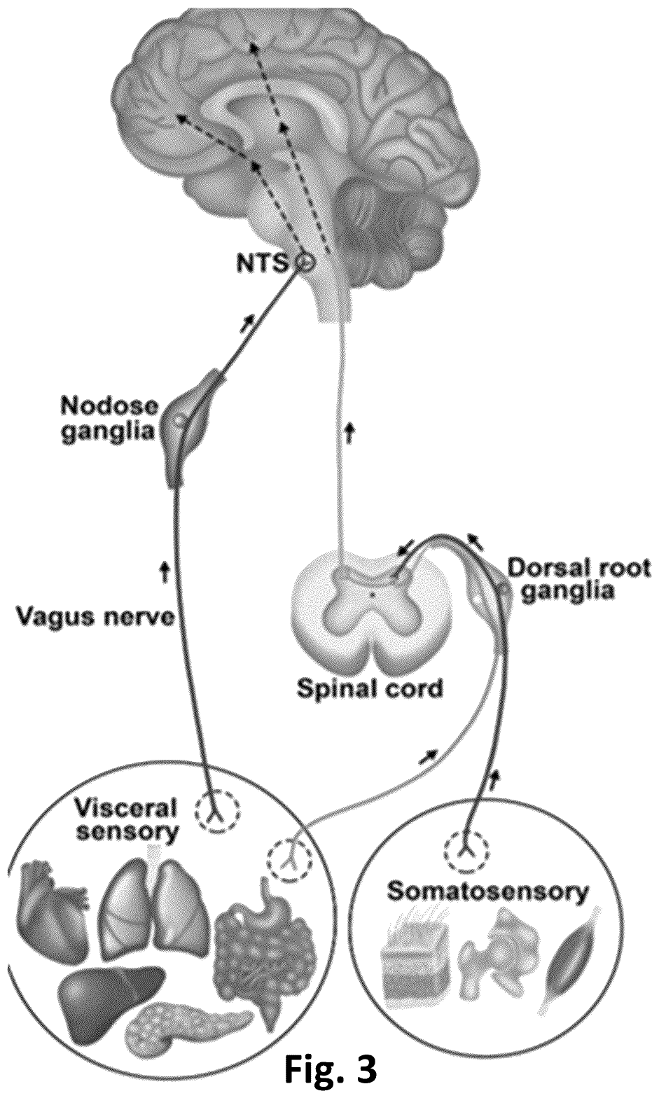

FIG. 3 shows how both somatic and autonomic afferent fibers activate the autonomic system. Afferent fibers, activated by inflammation, join a pathway to the solitary nucleus and the dorsal motor nucleus of the vagus located in the brain stem. From there, fibers follow the noradrenergic or cholinergic pathway.

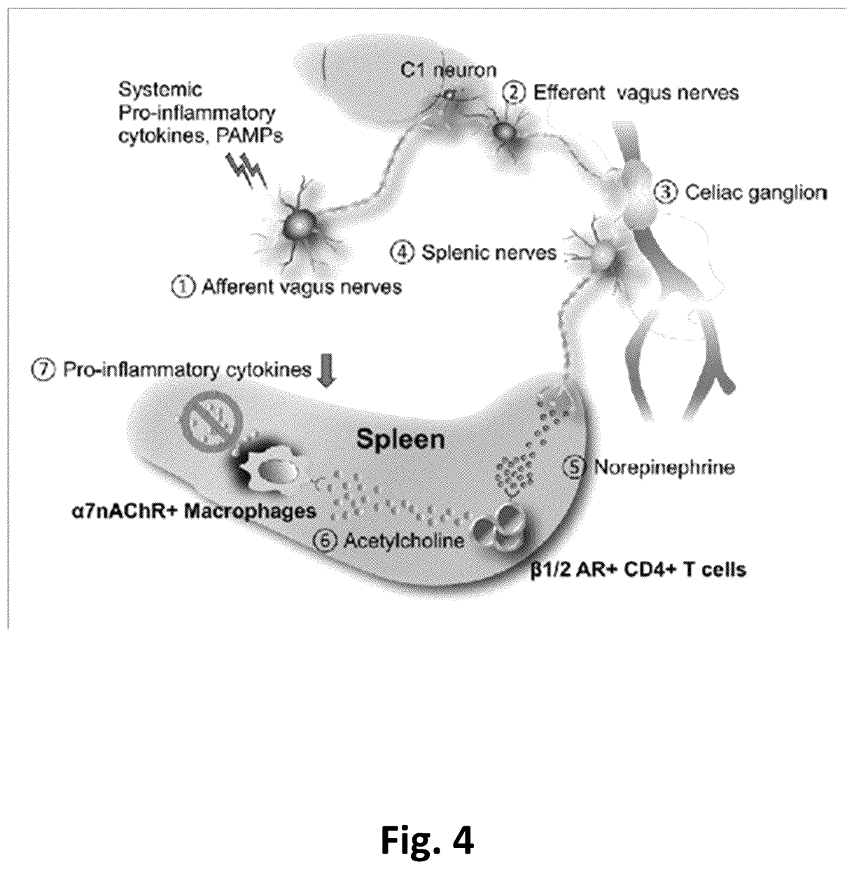

FIG. 4 shows the noradrenergic reflex system inhibiting inflammation. The Noradrenergic Reflex System transmits information about inflammation to the dorsal motor nucleus of the vagus which then sends pre-ganglionic efferent fibers in the vagus nerve to the celiac ganglion. The neurons of the celiac ganglion receive input from both the vagus (parasympathetic) and splanchnic (sympathetic) efferent nerves. The celiac ganglion sends post-ganglionic axons in the splenic nerve to reach the spleen. In the spleen, these axon terminals release norepinephrine, that in turn causes the release of acetylcholine from T lymphocytes. Acetylcholine suppresses the secretion of pro-inflammatory cytokines, such as TNFa by the M1 macrophages.

The Cholinergic Reflex System involves the motor nuclei of the vagus. These preganglionic efferent fibers travel in the vagus nerve to reach post-ganglionic ganglia within all internal organs. The terminals of these post-ganglionic fibers secrete acetylcholine within each organ to decrease the secretion of pro-inflammatory cytokines by M1 macrophages.

The Neuro-endocrine Reflex is activated by somatic and autonomic afferents responding to inflammations, stimulate the hypothalamus to secrete corticotrophin-releasing hormone in the portal system to the anterior pituitary gland. This initiates the secretion of adrenocorticotrophin hormone (“ACTH”) by pituitary corticotropes into the blood stream. ACTH stimulates adrenal cortical cells to secrete glucocorticoids, including cortisol. These glucocorticoids act to decrease the inflammatory response.

The Immuno-Axonal response occurs solely within the pain axons of the dorsal root ganglion cells without making any synaptic connections. Inflammatory peptides make holes in the axon which then allow the influx of calcium into the nerve itself. This influx causes the fiber to secrete molecules (CGRP, galanin, somatostatin, glutamate) at their terminals. Glutamate inhibits the inflammatory response in white blood cells.

### Reduction of Inflammation by Electrical Stimulation at a Single Site

Electrical stimulation at the site of afferent autonomic or somatic nerves inhibits inflammation and decreases the production of pro-inflammatory cytokines. Autonomic reflexes operate when vagal or somatic afferents are stimulated.

In an example, electrical stimulation of vagal afferents activates the hypothalamicuitary axis. This activation causes the release of adrenocortico-activating hormone from the hypothalamus. This activation results in the release of glucocorticoids to decrease inflammation.

In an example, electrical stimulation of vagal afferents affects the noradrenergic system. The sympathetic nerves release norepinephrine which stimulates splenic macrophages, to release acetylcholine. Acetylcholine suppresses the production of pro-inflammatory cytokines.

In an example, electrical stimulation of vagal afferents to the dorsal motor nucleus of the vagus results in the stimulation of vagal efferents. This pathway leads to the release of acetylcholine by post-ganglionic fibers. Acetylcholine decreases the production of pro-inflammatory cytokines, such as TNFa, by M1 macrophages. This cytokine participates in turning off the monocyte/macrophage system, thus limiting inflammation.

In an example, at a local site of inflammation, electrical stimulation of somatic afferents results in the same axon secreting a neurotransmitter to modulate the process of inflammation at the same local site by stimulating local cells, such as M2 macrophages, to release anti-inflammatory cytokines.

### Simultaneous TNA Device Application at Both Somatic and Autonomic Sites

In contrast to applying electrical stimulation at a single site to reduce inflammation, embodiments simultaneously apply electrical stimulation at both somatic and autonomic sites, which leads to unexpected results in reducing inflammation. In embodiments, a first patch 110 (“Patch A”) is applied to the person at or near the site of local inflammation to stimulate afferent nerves (somatic or autonomic) which activates one or more of the pathways described above, and a second patch 110 (“Patch B”) is applied to the person at or near the vagus nerve (including the auricular branch or other branches of the vagus nerve) to activate one or more of the pathways disclosed above.

Patch A and Patch B are stimulated simultaneously. Synchronicity of stimulation pulses between the two devices is not required but is allowed in embodiments. The coincident stimulation elicits anti-inflammation response in the person's tissues through multiple pathways at the same time, thereby reducing inflammation to a higher degree than either local or systemic mechanisms alone (i.e., unexpected results).

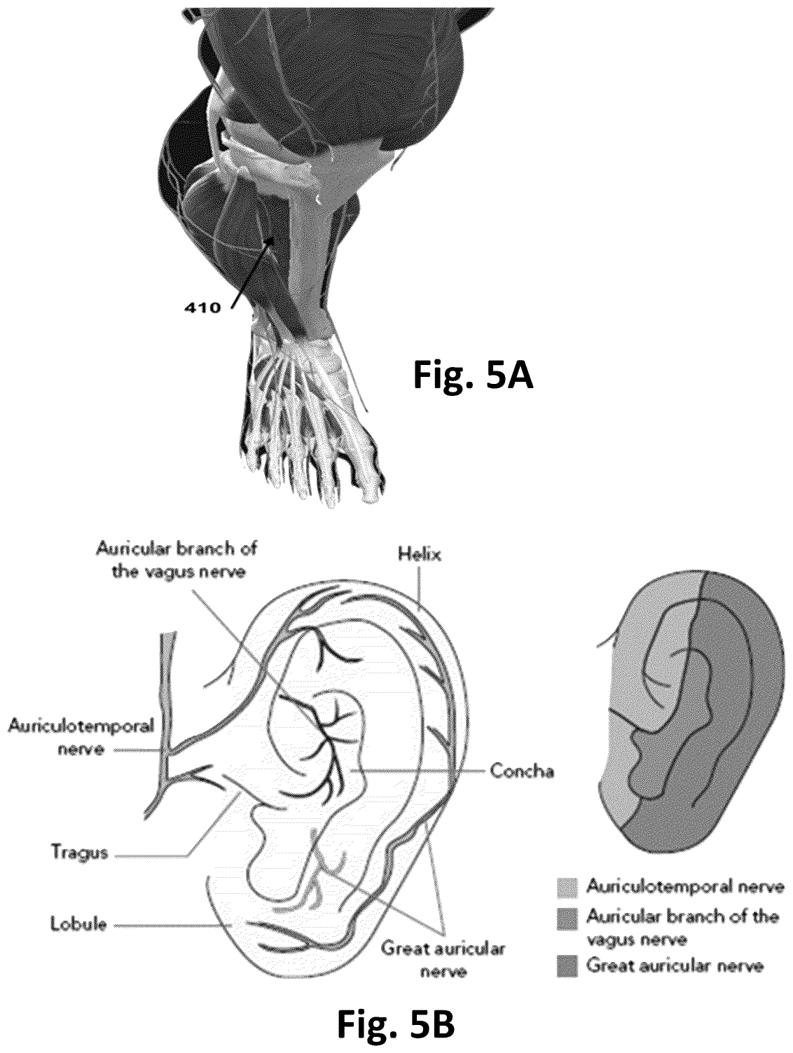

FIG. 5A shows the site of electrical stimulation of the common peroneal nerve in the lower leg, to inhibit inflammation in accordance to embodiments. In an example, Patch A is affixed to a person on the lower leg, over the common peroneal nerve, on or near an inflammation site. This is referred to as location ST36 in electroacupuncture. Stimulation of this nerve is shown in FIG. 5A. The Common Peroneal Nerve 410 is stimulated by the TNA Device 110.

FIG. 5B shows the site of electrical stimulation of the auricular branch of the vagus nerve to inhibit inflammation in accordance to embodiments. Patch B is affixed to a person near the ear, over the auricular branch of the vagus nerve, as shown in FIG. 5B or over another superficial autonomic site. In an example, Patch A is affixed to a person on the lower leg, over the tibial nerve, on or near an inflammation site. Patch B is affixed to a person near the ear, over the auricular branch of the vagus nerve, as shown in FIG. 5B or over another superficial autonomic site.

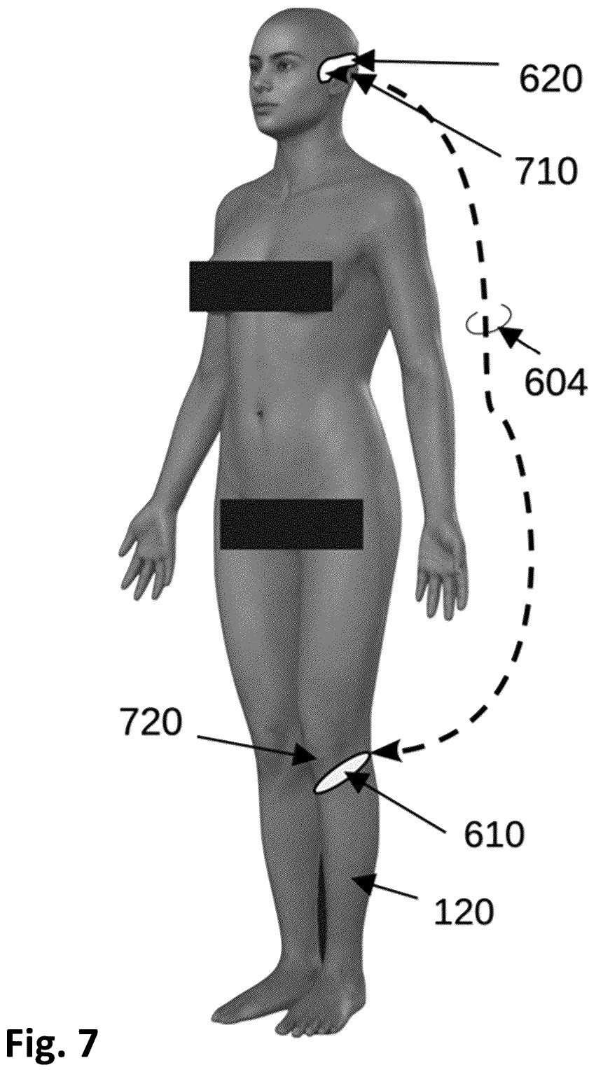

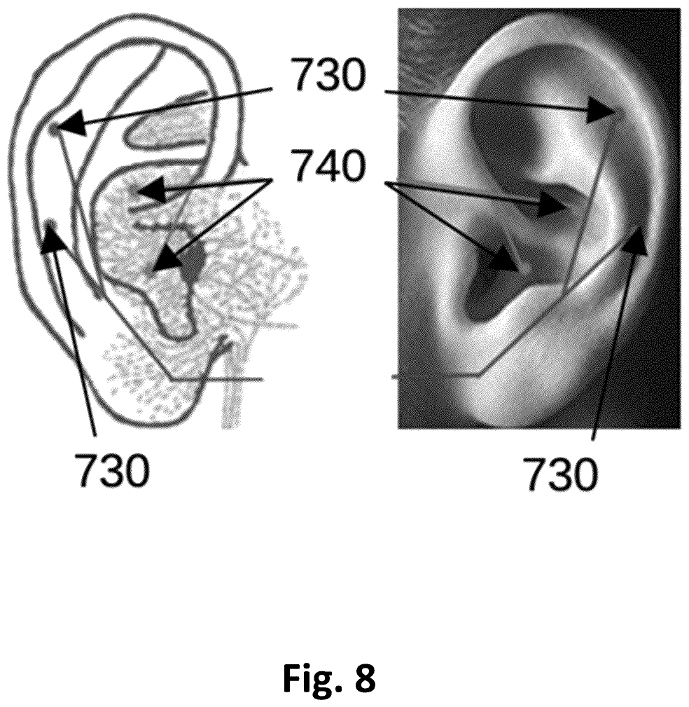

In an example, Patch A is affixed to a person on the lower leg, over the lateral sural cutaneous nerve, on or near an inflammation site. Patch B is affixed to a person near the ear, over the auricular branch of the vagus nerve (as shown in FIG. 7) or over another superficial autonomic site. The placement of Patch A and Patch B simultaneously will reinforce the anti-inflammatory effects of each other in an unexpected manner as follows: (1) there is more incoming information to the efferent system; and (2) activating the autonomic system will increase the anti-inflammatory response at the local site through a number of different processes, primarily through the activation of the M1 macrophage. This will result in an anti-inflammatory response greater than either A or B alone, or a linear combination of A plus B.

### Closed-Loop Feedback

In embodiments, electrical stimulation via patch 110 may be applied at one or more sites on the skin of the person or patient. A treatment or treatment protocol is the combination of electrical stimulation including amplitude, duration, time between stimulation sessions, pulse width, pulse frequency and other parameters defining the stimulation of the skin.

The one or more patches 110 may in some implementations communicate with one or more sensor, each sensor reading information from the person under treatment. Examples of sensor data may include body temperature at the site of the sensor, skin color or discoloration at the site of the sensor, chemical changes on the surface of the skin at the site of the sensor, blood flow, blood pressure, pulse rate, inflammatory markers such as cytokines, levels of nerve activity, etc. This communication provides a closed-loop mechanism for monitoring the treatment site and adapting the treatment protocol.

The purpose of the sensor data is to assess the state of the inflammation being treated and/or the state of the user's body, so that the treatment may be modified according to the values of sensor data. For example, an elevated skin temperature measured by a sensor at the site of inflammation may indicate infection, in which case the treatment should be stopped and the patient notified. For example, the perimeter of the inflammation may be detected by chemical, thermal, color means, in which case the treatment may be adjusted to stimulate a different region on the skin. For example, a decreasing surface temperature at the site of inflammation will signal a reduction in inflammation and thus, through this feedback, the treatment may be modified in intensity or duration to accommodate the reduced inflammation.

The one or more patches 110 read data from these sensors and adjust or adapt the treatment regime to optimize its effectiveness in reducing inflammation, to optimize the duration of treatment possible with the one or more devices before any need to be replaced, to optimize the level of comfort of the patient, etc. The treatment adjustment may be to one of the treatment devices, or to more than one of the treatment devices sequentially or simultaneously.

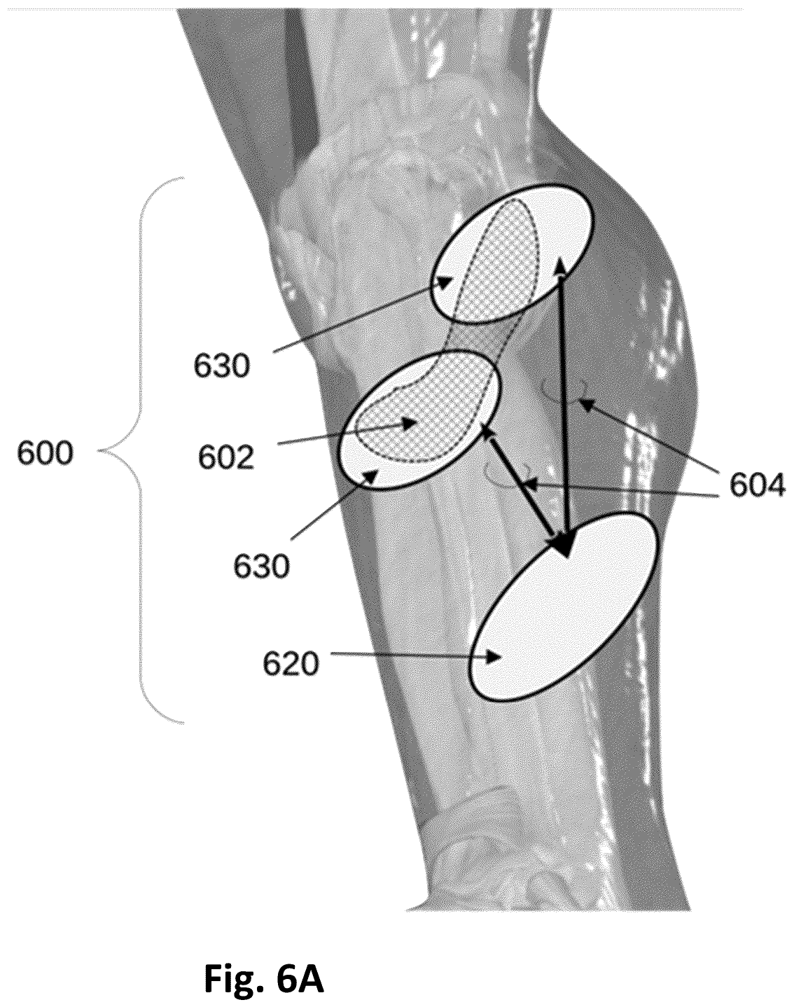

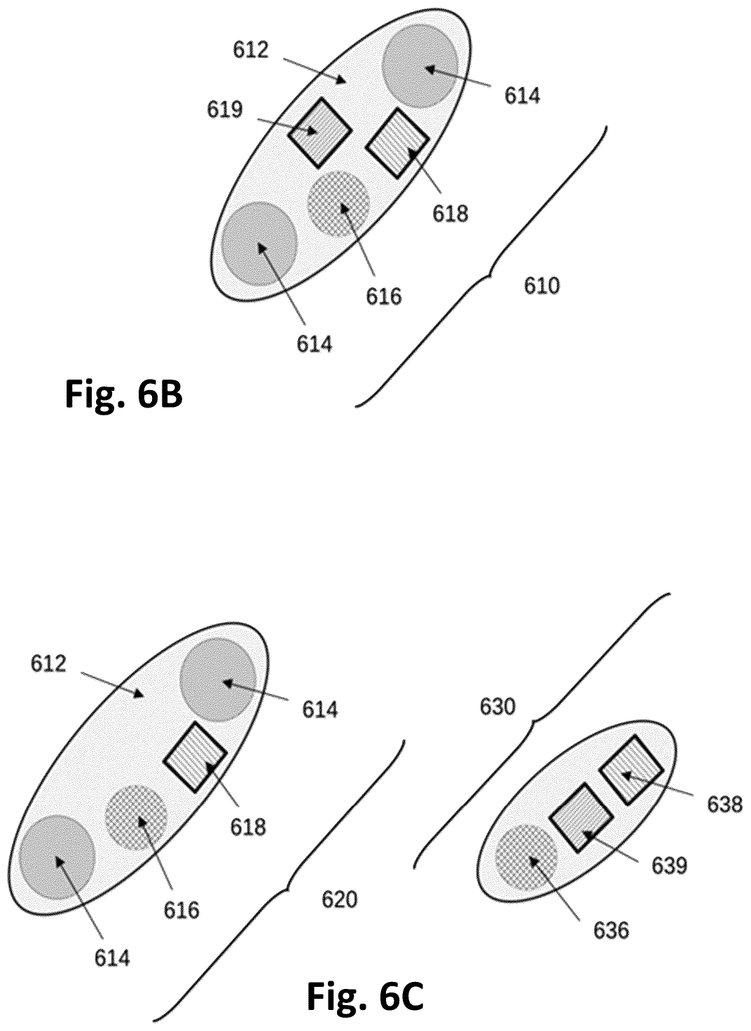

The one or more sensors may be placed directly on the one or more patches 110 as part of the functional design of such devices. For example, FIG. 6A shows a TNA Device System 600 applied to the skin, with one Paired TNA Device 620, and two Paired Sensor Devices 630, each applied on top of the Inflammation Site 602, and each Paired Sensor Device connected bi-directionally with the Paired TNA Device on a Feedback Channel 604, in accordance to embodiments. FIG. 6B shows a Smart TNA Device 610, with a securing mechanism 612, and one or more electrode pairs 614 with each pair having a positive electrode and a negative electrode, and a power source 616, and a processor 618, and a Sensor 619, in accordance to embodiments. FIG. 6C shows a Paired TNA Device 620, with a securing mechanism 612, and one or more electrode pairs 614 with each pair having a positive electrode and a negative electrode, and a power source 616, and a processor 618; and a Paired Sensor Device 630 with a power source 636, and a processor 638, and a Sensor 639, in accordance to embodiments.

Data from a Sensor 619 on one Smart TNA Device 610 may be communicated to other devices and may be communicated to a Processor 618 on the same device holding the Sensor.

The one or more sensors may be placed separately from the devices, such as on additional adhesively-affixed patches or belts or head coverings or other types of wound coverings, as shown in FIG. 6B. Data from a Paired Sensor Device 630, with a power source 636, and a processor 638, and a Sensor 639, may be sent to a Paired TNA Device 620.

The communication between sensor and device processor may be wired or wireless.

A set of one or more Smart TNA Devices 610 may communicate over a body area network with one or more Paired Sensor Device 630 to collect data from the one or more sensors.

A set of one or more Paired TNA Devices 620 may communicate over a body area network with one or more Paired Sensor Devices 630 to collect data from the one or more sensors.

An example, the Smart TNA Devices 610 are designed in shapes and sizes not specific to a particular User. The Smart TNA Devices are placed in locations over the Inflammation Site 602 and, as standard sized devices, may not treat all of the Inflammation Site at one time. The treatment plan may specify adjustments of the placement in order to treat the entire Inflammation Site.

An example, the Smart TNA Devices 610 are designed in shapes and sizes specific to a particular User and that User's Inflammation Site 602. These Smart TNA Devices are placed over the Inflammation Site to treat the complete site at one time.

An example, the several Smart TNA Devices 610 use differing treatment protocols, each controlled by the Paired TNA Device 620. For example, different positions on the Inflammation Site 602 may require different treatment protocols. The various treatment protocols may be defined by the Controller 618 at the beginning of the treatment period. The various treatment protocols may be adjusted by the Controller without regard for information from the Smart TNA Devices' Feedback Channel 604 data. The various treatment protocols may be adjusted by the Controller by incorporating information from the Smart TNA Devices' Feedback Channel 604 data.

The feedback loop in the TNA Device System 600 includes bi-directional Feedback Channels 604 between one or more Paired TNA Device 620 and one or more Smart TNA Device 610. The feedback control may be active when the devices are first applied to the User, or may be active for some or all of the period when the devices are on the User.

An example, the feedback loop through the Feedback Channels 604 is controlled by the User or a healthcare professional, such as wirelessly or by configuring the Paired TNA Device and/or the Smart TNA Device before applying it to the body.

An example, the feedback loop through the Feedback Channels 604 is controlled by the Controller 618 on the Smart TNA Device 610.

An example, the Controller 618 on the Smart TNA Device 610 uses an algorithm to monitor feedback data and adjust the treatment.

An example, the Controller 618 on the Smart TNA Device 610 uses a circuit to monitor feedback data and adjust the treatment.

An example, the Controller 618 on the Smart TNA Device 610 uses information sent to it from an external or remote system to monitor feedback data and adjust the treatment, such as from the internet from a medical professional who is monitoring the inflammation treatment.

An example, the sensor data from the one or more Paired TNA Devices 630 is recorded into a memory which may be local to the Smart TNA Device 620, or remote in a computer, server, or cloud-based system.

An example, the treatment settings data from the one or more Smart TNA Devices 620 is recorded into a memory which may be local to the one or more Smart TNA Devices, or remote in a computer, server, or cloud-based system.

An example, sensor data and/or treatment data is collected and analyzed by machine learning (“ML”) or other analytics to provide adaptive treatment to the User. The ML can be implemented on one or more of a processor of the patch, a processor in the on-body system, or a remote processor, either locally or remote (e.g., on a cloud infrastructure).

An example, the adaptive treatment is applied to the TNA Device System 600 in real time.

An example, the adaptive treatment is applied to future uses of the TNA Device System 600 on the same or on other Users.

An example, the sensor data and/or treatment data is collected into a file system and/or into a database such that treatment protocols may be calculated across a population of Users. Analysis may also derive conclusions regarding the efficacy of the inflammation treatment.

An example, the Paired TNA Device 620 indicates to the User that one or more Smart TNA Device 630 needs to be replaced and/or recharged to continue treatment. This indication to the User may be through a signal, such as a visual or audio signal, from the Paired TNA Device, or it may be through a notification in a connected smart device, such as a smart phone, laptop, tablet, smart watch, etc. The Paired TNA Device establishes a Feedback Channel 604 connection with the one or more replaced Smart TNA Device when the Smart TNA Device is activated.

An example, similar to the one above, the one or more Smart TNA Devices 630 indicates to the User that the Paired TNA Device 620 needs to be replaced and/or recharged to continue treatment. Treatment may be continued until the replaced Paired TNA Device is activated, or treatment may be paused until the replaced Paired TNA Device is activated.

FIG. 7 shows a User 120 with a Paired TNA Device 620, and a Smart TNA Device 610 in accordance to embodiments.

FIG. 8 shows the human ear in detail, with the Non-Auricular Vagus Nerve Stimulation Sites (taVNS) 730 and the Auricular Vagus Nerve Stimulation Sites (staVNS) 740.

As an example, shown in FIGS. 7 and 8, the User 120 uses both a Smart TNA Device 610 affixed below the knee 720, and a Paired TNA Device 620 affixed to the Auricular Vagus Nerve 710. The Paired TNA Device stimulates the Auricular Vagus Nerve at one or more of the Auricular Vagus Nerve Stimulation Sites 740 to reduce systemic inflammation. The Smart TNA Device monitors the degree of local inflammation using one or more sensor while also stimulating the nerve at the knee to reduce local inflammation. The two devices pass information in one or both directions along the Feedback Channel 604.

Several example inventions are specifically illustrated and/or described herein. However, it will be appreciated that modifications and variations of the disclosed example inventions are covered by the above teachings and within the purview of the appended claims without departing from the spirit and intended scope of the invention.

What is claimed is:

1. A method of treating inflammation of a user, the method comprising:
applying a first patch on a dermis of the user adjacent to an afferent nerve, the first patch comprising first electrodes;
applying a second patch on the dermis of the user adjacent to a vagus nerve or a vagus nerve connecting branch, the second patch comprising second electrodes; and
generating a treatment protocol during a time period comprising simultaneously applying a first electrical stimuli to the afferent nerve via the first electrodes of the first patch and applying a second electrical stimuli to the vagus nerve or the vagus nerve connecting branch via the second electrodes of the second patch.

2. The method of claim 1, wherein the first patch is applied on or near an occurrence of the inflammation.

3. The method of claim 1, wherein the vagus nerve connecting branch comprises at least one of a peroneal nerve, a tibial nerve, a lateral sural cutaneous nerve, a medial sural cutaneous nerve or an auricular branch of the vagus nerve.

4. The method of claim 1, wherein the second patch is applied on a lower leg of the user.

5. The method of claim 1, wherein the second patch is applied on or near an ear of the user.

6. The method of claim 1, wherein the applying the first electrical stimuli is synchronous with the applying the second electrical stimuli.

7. The method of claim 1, further comprising:
after the treatment protocol, sensing a state of the inflammation; and
based on the state, generating a subsequent treatment protocol comprising different intensity or duration from a previous treatment protocol.

8. The method of claim 7, further comprising using machine learning to sense the state of the inflammation.

9. The method of claim 7, wherein the sensing the state of the inflammation comprising using one or more sensors that are included on the first or second patches.

10. The method of claim 7, wherein the sensing the state of the inflammation comprising using one or more sensors that are separate from the first or second patches.

11. An inflammation treatment system comprising:
a first patch adapted to be externally coupled on a dermis of a user adjacent to an afferent nerve, the first patch comprising first electrodes;
a second patch adapted to be externally coupled on the dermis of the user adjacent to a vagus nerve or a vagus nerve connecting branch, the second patch comprising second electrodes; and
one or more processors adapted to generate a treatment protocol during a time period comprising simultaneously applying a first electrical stimuli to the afferent nerve via the first electrodes of the first patch and applying a second electrical stimuli to the vagus nerve or the vagus nerve connecting branch via the second electrodes of the second patch.

12. The inflammation treatment system of claim 11, wherein the first patch is applied on or near an occurrence of the inflammation.

13. The inflammation treatment system of claim 11, wherein the vagus nerve connecting branch comprises at least one of a peroneal nerve, a tibial nerve, a lateral sural cutaneous nerve, a medial sural cutaneous nerve or an auricular branch of the vagus nerve.

14. The inflammation treatment system of claim 11, wherein the second patch is applied on a lower leg of the user.

15. The inflammation treatment system of claim 11, wherein the second patch is applied on or near an ear of the user.

16. The inflammation treatment system of claim 11, wherein the applying the first electrical stimuli is synchronous with the applying the second electrical stimuli.

17. The inflammation treatment system of claim 11, the processor further adapted to:
after the treatment protocol, sensing a state of the inflammation; and
based on the state, generating a subsequent treatment protocol comprising different intensity or duration from a previous treatment protocol.

18. The inflammation treatment of claim 17, the processor further adapted to:
using machine learning to sense the state of the inflammation.

19. The inflammation treatment of claim 17, wherein the sensing the state of the inflammation comprising using one or more sensors that are included on the first or second patches.

20. The inflammation treatment of claim 17, wherein the sensing the state of the inflammation comprising using one or more sensors that are separate from the first or second patches.
